(19) United States
(12) Patent Application Publication
(10) Pub. No.: US 2025/0333683 A1
(43) Pub. Date: Oct. 30, 2025

(54) A METHOD AND SYSTEM FOR SIMULATING STEM CELL DIFFERENTIATION DYNAMICS

(71) Applicant: CellVoyant Technologies Limited, Bristol (GB)

(72) Inventors: Vladimir Slaykovskiy, Bristol (GB); Edward Ren, Bristol (GB); Rickard Sjögren, Bristol (GB)

(21) Appl. No.: 19/190,195

(22) Filed: Apr. 25, 2025

Related U.S. Application Data
(60) Provisional application No. 63/639,136, filed on Apr. 26, 2024.

(51) Int. Cl. C12M 1/36; C12M 1/34; G06F 30/27

(52) U.S. Cl. CPC C12M 41/48; C12M 41/46; G06F 30/27

(57) ABSTRACT

Provided herein are systems and a computer-implemented methods for predicting and/or simulating stem cell differentiation dynamics, including optimizing stem cell differentiation dynamics based on real-time monitoring of a stem cell culture undergoing differentiation.

### CROSS REFERENCE TO RELATED APPLICATION

This application claims priority to U.S. Provisional Application 63/639,136, filed Apr. 26, 2024, the disclosure of which is herein incorporated by reference in its entirety.

### TECHNICAL FIELD

The present disclosure relates generally to a system, an apparatus, and a computer-implemented method for predicting and/or simulating a future cell culture state based on a current state and one or more actions and, more particularly for predicting and/or simulating stem cell differentiation dynamics, including optimizing stem cell differentiation dynamics based on real-time monitoring of a stem cell culture undergoing differentiation.

### BACKGROUND

Stem cell differentiation efficiency exhibits a notable degree of inconsistency among different cell lines and donors. For instance, gene expression of stem cells differs between lines. This inconsistency has prompted researchers to assess the shortcomings of traditional static differentiation protocols, which often yield suboptimal results due to their inherent inflexibility.

To address this issue, there is growing interest in the concept of dynamic differentiation protocols. These protocols, in theory, involve the adjustment of timings and concentrations of added growth factors based on the current state of the cell culture. This may lead to improved outcomes, although the exact efficacy remains uncertain.

A key challenge in implementing dynamic protocols lies in the need to forecast the outcomes of various scenarios accurately. The goal is to determine how changes in protocol parameters such as timings and concentrations may impact the course of stem cell differentiation. Accurate forecasting could enable the selection of more suitable differentiation strategies.

### SUMMARY

The instant disclosure provides an innovative technological solution that includes a hybrid model, combining mechanistic modelling and machine learning, to simulate the future state of a cell culture undergoing differentiation given initial conditions, real-time measurements, and past and future perturbations to the cell culture. The solution includes a computer-implemented method that comprises training a continuous-time machine learning model to predict the rate of change of cellular subpopulations in the cell culture, and using the trained model to simulate the time-trajectory of how cellular subpopulations change to either predict future timepoints or interpolate between discrete measurements.

According to an aspect of the disclosure, a computer-implemented method is provided for optimizing stem cell differentiation based on real-time monitoring of a stem cell culture undergoing differentiation. The method comprises: receiving, by a processor, a request for a yield of a target cell type i at a specific time τ in the future, and receiving, by the processor, one or more measurements of a cell culture containing n discrete states of cells undergoing cell differentiation at time t, where n is a positive integer. The one or more measurements can include any type of measurement of a cell state, including, for example, but not limited, to inline or online process parameters (pH, temperature, glucose, lactate, dissolved oxygen, spectroscopy, conductivity, optical density, capacitance, medium viscosity, redox potential, mass spectrometry, fluid density), or online or atline phenotypic measurement (imaging, flow cytometry). The method further comprises providing, by the processor, the one or more measurements of the cell culture to a machine learning platform; generating, by the machine learning platform, a mathematical representation for each of the n discrete states based on the one or more measurements, the mathematical representation including a current state x(t) and an action α(t) for each of the n discrete states at time t, including a current state xᵢ(t) and an action αᵢ(t) for the target cell type i; transforming for each of the n discrete states, by the machine learning system, the current state x(t) and the action α(t) to a state change rate r_{θ}, where θ is a parametric weight value mapped by the machine learning platform to the current state x(t) and the action α(t) for each of the n discrete states; receiving, by a mechanistic platform, the state change rate r_{θ}; calculating, by the mechanistic platform, a state differential rate dx/dt for each of the n discrete states, including a state differential rate dxᵢ/dt for the target cell type i, based on the state change rate r_{θ}; integrating, by the mechanistic platform, the state differential rate dx/dt for each of the n discrete states from an initial time tₒ to the time τ to calculate the yield of the target cell type i at time τ; and outputting the calculated yield of the target cell type i for the time τ.

The method can comprise displaying the calculated yield on a display device.

The method can comprise optimizing the calculated yield under alternative actions α_{ALT}(t); and outputting an optimized yield based on the alternative actions α_{ALT}( ) The optimizing can include receiving one or more alternative actions α_{ALT}(t) and repeating steps (d) to (i) of the method. The one or more alternative actions α_{ALT}(t) can be received from a human interface device.

The method can include outputting the calculated yield via a human interface device. The human interface device can include a user interface, a mobile device, a display device, or a computing device.

The method can comprise optimizing the yield for the target cell type i for the time τ, wherein the optimizing comprises calculating the yield under different actions to find an optimal action to take. The method can comprise displaying the optimized yield on a display device or transmitting the optimized yield to a computing device.

According to another aspect of the disclosure, a non-transitory computer readable storage medium is provided, storing one or more programs comprising instructions, which, when executed by a processor, perform: (a) receiving, by the processor, a request for a yield of a target cell type i at a specific time τ in the future; (b) receiving, by the processor, one or more measurements of a cell culture containing n discrete states of cells undergoing cell differentiation at time t, where n is a positive integer; (c) providing, by the processor, the one or more measurements of the cell culture to a machine learning platform; (d) generating, by the machine learning platform, a mathematical representation for each of the n discrete states based on the one or more measurements, the mathematical representation including a current state x(t) and an action α(t) for each of the n discrete states at time t, including a current state xᵢ(t) and an action αᵢ(t) for the target cell type i; (e) transforming for each of the n discrete states, by the machine learning system, the current state x(t) and the action (t) to a state change rate r_{θ}, where θ is a parametric weight value mapped by the machine learning platform to the current state x(t) and the action α(t) for each of the n discrete states; (f) receiving, by a mechanistic platform, the state change rate r_{θ}; (g) calculating, by the mechanistic platform, a state differential rate dx/dt for each of the n discrete states, including a state differential rate dxᵢ/dt for the target cell type i, based on the state change rate r_{θ}; (h) integrating, by the mechanistic platform, the state differential rate dx/dt for each of the n discrete states from an initial time t₀ to the time τ to calculate the yield of the target cell type i at time τ; and (i) outputting the calculated yield of the target cell type i for the time τ.

The one or more programs can comprise instructions, which, when executed by the processor, perform: displaying the calculated yield on a display device.

The one or more programs can comprise instructions, which, when executed by the processor, perform: optimizing the calculated yield under alternative actions α_{ALT}(t); and outputting an optimized yield based on the alternative actions α_{ALT}(t).

The one or more programs can comprise instructions, which, when executed by the processor, perform: receiving, by the processor, one or more alternative actions α_{ALT}(t); and repeating steps (d) to (i). The one or more alternative actions α_{ALT}(t) can be received from a human interface device.

According to a further aspect of the disclosure, an apparatus is provided for optimizing a stem cell differentiation based on real-time monitoring of the stem cell culture undergoing differentiation using live cell measurements. The apparatus comprises: one or more input devices; one or more output devices including a human interface device; a machine learning platform; a mechanistic platform including an integrator; one or more processors; and a memory storing one or more programs to be executed by the one or more processors. The one or more programs comprise instructions for: receiving a request for a yield of a target cell type i at a specific time τ in the future; receiving one or more measurements of a cell culture containing n discrete states of cells undergoing cell differentiation at time t, where n is a positive integer; providing the one or more measurements of the cell culture to the machine learning platform; generating, by the machine learning platform, a mathematical representation for each of the n discrete states based on the one or more measurements, the mathematical representation including a current state x(t) and an action α(t) for each of the n discrete states at time t including a current state x(t) and an action αᵢ(t) for the target cell type i; transforming for each of the n discrete states, by the machine learning system, the current state x(t) and the action α(t) to a state change rate r_{θ}, where θ is a parametric weight value mapped by the machine learning platform to the current state x(t) and the action α(t) for each of the n discrete states; receiving, by the mechanistic platform, the state change rate r_{θ}; calculating, by the mechanistic platform, a state differential rate dx/dt for each of the n discrete states, including a state differential rate dxᵢ/dt for the target cell type i, based on the state change rate r_{θ}; integrating, by the integrator, the state differential rate dx/dt for each of the n discrete states from an initial time tₒ to the time τ to calculate the yield of the target cell type i at time τ; and outputting the calculated yield of the target cell type i for the time τ. The one or more programs comprise instructions for receiving the one or more alternative actions α_{ALT}(t) from a human interface device and calculating the optimized yield based on the alternative actions α_{ALT}(t).

The apparatus can comprise a display device configured to display the calculated yield.

The apparatus can comprise a transmitter configured to send the calculated yield to a computing device.

The apparatus can comprise a human interface device configured to receive one or more alternative actions α_{ALT}(t).

The apparatus can be configured to optimize the calculated yield under alternative actions α_{ALT}(t) and output an optimized yield based on the alternative actions α_{ALT}(t).

The one or more measurements can comprise at least one of an inline process parameter or an online process parameter. The inline process parameter or the online process parameter can comprise at least one of a pH measurement value, a temperature measurement value, a glucose measurement value, a lactate measurement value, a dissolved oxygen measurement value, a spectroscopy measurement value, a conductivity measurement value, an optical density measurement value, a capacitance measurement value, a medium viscosity measurement value, a redox potential measurement value, a mass spectrometry measurement value, and/or an ultrasound-based measurement value of fluid density.

The one or more measurements can comprise an online phenotypic measurement or an atline phenotypic measurement. The online phenotypic measurement or the atline phenotypic measurement can comprise one or more images or flow cytometry.

The stem cell culture can be selected from an embryonic stem cell culture, an adult stem cell culture, an induced pluripotent stem cell culture, or a trophoblast stem cell culture.

The stem cell culture can include a mesenchymal stem cell culture, a hematopoietic stem cell culture, a neural stem cell culture, an epithelial stem cell culture, or a cord blood stem cell culture.

The stem cell culture can comprise progenitor cells. The progenitor cells can be selected from the group consisting of mesodermal progenitor cells, endodermal progenitor cells, ectodermal progenitor cells, neural progenitor cells, cardiac progenitor cells, hematopoietic progenitor cells, mesenchymal stem cells, pancreatic progenitor cells, and a combination thereof.

The stem cell culture undergoing differentiation can result in the stem cell culture differentiating into a mesoderm, endoderm, and/or ectoderm.

The mesoderm can comprise a skeletal muscle cell, a cardiac muscle cell (i.e., a cardiomyocyte), a kidney cell, a red blood cell, or a smooth muscle cell. The endoderm can comprise a lung cell, a thyroid cell, or a pancreatic cell. The ectoderm can comprise a skin cell, a neuron cell, or a pigment cell.

The yield of a target cell type can comprise an amount of the target cell type, a level of growth of the target cell type, and/or a specific composition of the subpopulations of the target cell type.

The discrete states of stem cells undergoing differentiation can be selected from a specific cell type or subtype or a specific fate of the cell.

The action can be selected from the group consisting of maintaining the cell culture state, modulating the cell culture state, ending the cell culture, requesting further measurements, or requesting complementary measurements.

The machine learning platform can comprise a neural network.

The mechanistic platform can comprise an application specific integrated circuit (ASIC).

The mechanistic platform can comprise one or more programs that are executed by the processor.

Additional features, advantages, and embodiments of the disclosure may be set forth or apparent from consideration of the detailed description and drawings. Moreover, it is to be understood that the foregoing summary of the disclosure and the following detailed description and drawings provide non-limiting examples that are intended to provide further explanation without limiting the scope of the disclosure as claimed.

### BRIEF DESCRIPTION OF THE DRAWINGS

The accompanying drawings, which are included to provide a further understanding of the disclosure, are incorporated in and constitute a part of this specification, illustrate embodiments of the disclosure and together with the detailed description serve to explain the principles of the disclosure. No attempt is made to show structural details of the disclosure in more detail than may be necessary for a fundamental understanding of the disclosure and the various ways in which it may be practiced.

FIG. 1 shows a block diagram of a system for forecasting and/or simulating a cell culture state at one or more points in time, including predicting and/or simulating a yield of a target cell type in the cell culture.

FIG. 2 shows a block diagram of an embodiment of an apparatus for predicting and simulating cell differentiation dynamics.

FIG. 3 shows a block diagram of a hybrid model implementation in the system of FIG. 1.

FIG. 4 shows a process for predicting and simulating cell differentiation dynamics.

FIG. 5 shows a schematic representation of the experimental plan provided in the Examples section.

FIG. 6 shows a chart demonstrating the Neural network predicted transition rate matrix between different fates at a CHIR99021 concentration of 0.93 μM. Cells were predicted to transition from the unknown fate to the mesoderm fate at a rate of 1.4%/h.

FIG. 7 shows a FateSim simulated fate evolution graph compared against observed ground truth fate evolutions. FateSim correctly predicted that the CHIR99021 dosage was insufficient to drive a transition of pluripotent cells towards the mesoderm lineage.

FIG. 8 shows a chart demonstrating the Neural network predicted transition rate matrix between different fates at a CHIR99021 concentration of 1.93 M. Cells were predicted to transition from the unknown fate to the mesoderm fate at a rate of 7.4%/h.

FIG. 9 shows a FateSim simulated fate evolution graph compared against observed ground truth fate evolutions after applying CHIR99021 at a concentration of 1.93 μM for 48 hours. FateSim correctly predicted that the CHIR99021 dosage drove a transition of pluripotent cells towards the mesoderm lineage up until the removal of CHIR99021 after which there was a decline in mesoderm yield.

FIG. 10 shows a graph demonstrating a CHIR99021 drug response curve.

The present disclosure is further described in the detailed description that follows.

### DETAILED DESCRIPTION

The disclosure and its various features and advantageous details are explained more fully with reference to the non-limiting embodiments and examples that are described or illustrated in the accompanying drawings and detailed in the following description. It should be noted that features illustrated in the drawings are not necessarily drawn to scale, and features of one embodiment can be employed with other embodiments as those skilled in the art would recognize, even if not explicitly stated. Descriptions of well-known components and processing techniques may be omitted so as to not unnecessarily obscure the embodiments of the disclosure. The examples are intended merely to facilitate an understanding of ways in which the disclosure can be practiced and to further enable those skilled in the art to practice the embodiments of the disclosure. Accordingly, the examples and embodiments should not be construed as limiting the scope of the disclosure. Moreover, it is noted that like reference numerals represent similar parts throughout the several views of the drawings.

State-of-the-art solutions for modeling stem cell differentiation processes fall into two main categories. The first main category is based on growth modelling, whereas the population growth and death dynamics is modelled and forecasted. These models can be mechanistic, where the dynamics are described using differential equations, or data-driven, such as machine learning models. There are also hybrid models, which use a combination of mechanistic and data-driven models. Hybrid models are used in biomanufacturing of antibodies, but also have demonstrated utility in cell therapy manufacturing. These growth models provide no insight into how a cell state changes during differentiation due to a poor understanding of how process parameters link to differentiation processes.

The second main category models cell state changes on a molecular level using equation systems based on gene regulatory networks. Simpler approaches based on the expression of single transcription factors have been demonstrated in control of cell culture processes, such as, for example, in maintaining embryonic stem cell culture and improving differentiation. These approaches, however, are limited to modelling cellular states where the gene regulatory network is well understood. Using it during real-time forecasting of a cell culture process relies on being able to directly measure gene expression of key genes, such as, for instance, using genetically engineered cells with fluorescent markers. They are, therefore, not easily scalable to new use cases.

The instant disclosure provides a technological solution that simulates the population differentiation dynamics of a stem cell culture, using a hybrid model formulation. The solution includes a model that describes how a composition of discrete states evolve over time in a cell culture of mixed states. The cell culture can include a stem cell culture undergoing differentiation, in which case the discrete states are different cell types, subtypes, or fates. The cell subtypes may be the starting and target cell type or fate of differentiation, as well as any subtypes the cells may differentiate into during differentiation. The discrete states may also be quantified as discretized expression levels of one or more biomarkers, for instance gene expression of a transcription factor.

In an embodiment, a model of stem cell differentiation for a given cell culture with n possible cell states can have x: [symbol] → [symbol] ⁿ represent the absolute number, or proportion, of cells of each state at time point t. The rate function \hat{r}_{θ} can be denoted as \hat{r}_{θ}: [symbol] × [symbol] × [symbol] → [symbol] ⁺:

$${\hat{r}}_{θ} ( a , b , t ) = {\hat{f}}_{θ} ( a ( t ) , {F}_{b} , {F}_{a} , x ( t ) ) \qquad ( 1 )$$

where F∈ [symbol] ^{n×m} contains all cell state representations Fᵢ∈ [symbol] ^{m}, a: [symbol] → [symbol] ^{a} provides the action representation at time t and \hat{f}_{θ}: [symbol] ^{a×m×m×n}→ [symbol] ⁺ is a possibly non-linear function parameterized by e mapping the action, state and number of cells in all states to a rate. In contrast to fully mechanistic approaches, which could model the weights as a system of equations, such as, for example, describing transcription factor expression, the solution includes a data-driven component. For instance, in the solution \hat{f}_{θ} can be represented by a machine learning platform comprising, for example, an artificial neural network (ANN), with weights θ. An advantage of this approach is that the solution can model the rates of change without the requirement of fully knowing, or directly measure, the internal state of cells.

The rate of which the number of cells of state i change with respect to time can be described using a process master equation, as:

$$\frac{{dx}_{i}}{dt} = {\sum }_{j = 1 , j \neq i}^{n} {\hat{r}}_{{θ}_{ji}} ( i , j , t ) {x}_{j} ( t ) - {\sum }_{j = 1 , j \neq i}^{n} {\hat{r}}_{{θ}_{ij}} ( j , i , t ) {x}_{i} ( t ) + {\hat{r}}_{{θ}_{div}} ( i , i , t ) {x}_{i} ( t ) - {\hat{r}}_{{θ}_{δ}} ( i , i , t ) {x}_{i} ( t ) \qquad ( 2 )$$

where \hat{r}_{θ}_{ᵢⱼ} denotes the rates of cells changing state i→j, \hat{r}_{θ}_{ⱼᵢ} the rate of changing state j→i, \hat{r}_{θ}_{_{div}} the division rate and \hat{r}_{θ}_{_{δ}} the death rate.

Even though Equation (2) is a general formulation, it has two limitations. First, unless experimental measurements are provided for each cell changing between each of the states as well as cell death and division, the problem will be underspecified. Such measurements are much more difficult to obtain compared to cell counts of each state. As a result, there can exist, potentially, an infinite number of parameters that fit the pairs θⱼᵢ, θᵢⱼ and θ_{div}, θ_{δ}, which makes model fitting difficult. Second, unconstrained models can make it difficult to interpret the model since fitted rates may fit experimental data well but be biologically implausible. For instance, a net change rate of zero growth may be fitted by large division and death rate as well as zero rate of each.

To solve these limitations, the solution can include first replacing all rate functions, \hat{r}_{θ}, with a single, unbounded, net rate of change function r_{θ}: [symbol] × [symbol] → [symbol] , with f_{θ}: [symbol] ^{a×m×m×n}→ [symbol] , and let r_{θ}(i, i) represent the net growth rate:

$${\hat{r}}_{{θ}_{diν}} ( i , i , t ) {x}_{i} ( t ) - {\hat{r}}_{{θ}_{δ}} ( i , i , t ) {x}_{i} ( t ) = ( {\hat{r}}_{{θ}_{diν}} ( i , i , t ) - {\hat{r}}_{{θ}_{δ}} ( i , i , t ) ) {x}_{i} ( t ) := {r}_{θ} ( i , i , t ) {x}_{i} ( t ) \qquad ( 3 )$$

The solution can include applying an antisymmetric constraint to Equation (2) to reach the following model:

$$\frac{{dx}_{i}}{dt} = {g}_{i , θ} ( t , {x}_{i} ( t ) ) = {\sum }_{j = 1 , j \neq i}^{n} {r}_{θ} ( i , j , t ) {x}_{j} ( t ) - {\sum }_{j = 1 , j \neq i}^{n} {r}_{θ} ( j , i , t ) {x}_{i} ( t ) + {r}_{θ} ( i , i , t ) {x}_{i} ( t ) \qquad ( 4 )$$

$$s . t . {r}_{θ} ( i , j , t ) = - {r}_{θ} ( j , i , t ) ∀ j , j \neq i$$

In fitting a model from data, the solution can include modeling the rate f_{θ}, using the machine learning platform (for example, an ANN), which can include, for example, a non-linear and multi-layered model. In this regard, parameter estimation is challenging. To overcome this challenge, the solution includes employing a neural ordinary differential equation (NODE) methodology to train the model. In an embodiment, the solution uses dxᵢ/dt=g_{i,θ}(t,x(t)) as defined in Equation (4) to find the number of cells in state i at time t by evaluating the integral:

$${x}_{i} ( τ ) = {x}_{i} ( {t}_{0} ) + {\int }_{{t}_{0}}^{τ} {g}_{i , θ} ( t , {x}_{i} ( t ) ) dt \qquad ( 5 )$$

Computationally, the solution can use a numerical ODE-solver to integrate g_{i,θ} as:

$${x}_{i} ( τ ) = ODEsolve ( {x}_{i} ( {t}_{0} ) , {g}_{i , θ} , {t}_{0} , τ ) \qquad ( 6 )$$

As will be understood by those skilled in the art, an ODE solver is a computational tool that can be designed to analyze the dynamic relationships between variables over time. For example, an ODE solver can include Runge-Kutta methods or A dams-Bashforth methods; whereas the adjoint sensitivity method can be used for NODEs.

For a given experimental measurements \hat{x}ᵢ=<\hat{x}ᵢ(t₀), \hat{x}ᵢ(t₁), . . . , \hat{x}ᵢ(t_{d})>, which could be irregularly spaced in time, and differentiable loss function L: [symbol] × [symbol] → [symbol] and dxᵢ/dt=gᵢ(t) as defined in Equation (4), the solution can find the optimal set of parameters as given by:

$${θ}^{*} = {argmin}_{θ} {\sum }_{i = 1}^{n} {\sum }_{k = 0}^{d} L ( ODEsolve ( {x}_{i} ( {t}_{0} ) , {g}_{i , θ} , {t}_{0} , {t}_{k} ) , {\hat{x}}_{i} ( {t}_{k} ) ) \qquad ( 7 )$$

by updating θ iteratively using stochastic gradient descent and the back-propagation algorithm to compute the gradient backwards through the solver.

After model training, the solution can predict and/or simulate the number of cells of each state in a training dataset by integrating from the current time point into the future. Given an observation of the current state of the cell culture \hat{x}ᵢ(t_{now}), the solution can use the observed value as initial value in Equation (8):

$${x}_{i} ( τ ) = ODEsolve ( {\hat{x}}_{i} ( {t}_{now} ) , {g}_{i , {θ}^{*}} , {t}_{now} , τ ) \qquad ( 8 )$$

where τ is the simulation time horizon. To simulate complete experiments from start, the solution can set the starting cell states \hat{x}(t₀) to a known composition of cellular states, for example, but not limit to, 100% pluripotent stem cells.

The solution can restrict fate transition, such as, for example, based on prior knowledge. For instance, given a set of disallowed transitions C={(a, b)|a, b∈ [symbol] , 1≤a≤n, 1≤b≤n}, Equation (4) can be modified as follows:

$$\frac{{dx}_{i}}{dt} = {g}_{i , θ} ( t , {x}_{i} ( t ) ) = {\sum }_{j = 1 , j \neq i}^{n} {r}_{θ} ( i , j , t ) {x}_{j} ( t ) - {\sum }_{j = 1 , j \neq i}^{n} {r}_{θ} ( j , i , t ) {x}_{i} ( t ) + {r}_{θ} ( i , i , t ) {x}_{i} ( t ) \qquad ( 9 )$$

$$s . t . {r}_{θ} ( i , j , t ) = - {r}_{θ} ( j , i , t ) ∀ j , j \neq i , ( i , j ) ∉ C$$

$$s . t . {r}_{θ} ( i , j , t ) = 0 , ∀ ( i , j ) , ( i , j ) ∈ C$$

The solution can include additional process information. Whereas the formulation of the rate prediction incorporates the sequence of actions applied to a stem cell culture, it is limited to interventions applied during differentiation. The solution extends the rate function into:

$${f}_{θ} : {ℝ}^{a \times m \times m \times n \times s} \to ℝ \qquad ( 10 )$$

by taking the vector of initial culture state s₀∈ [symbol] ^{s} modifying Equation (1):

$${r}_{θ} ( a , b , t ) = {f}_{θ} ( a ( t ) , {F}_{b} , {F}_{a} , x ( t ) , {s}_{0} ) \qquad ( 11 )$$

Example initial conditions can include cell seeding density, cell line characteristics, culture vessel characteristics-such as size or surface coating—or passage number of cells.

The solution can represent state and actions. By using a machine learning (ML) platform to transform the state and action representation, the solution provides flexibility to how state and actions are represented mathematically. This enables the solution to exploit prior knowledge in several ways. The states and actions can be represented directly by, for example, indicator matrices, which include matrices whose elements are either one or zero, or weighted indicator matrices, which follow the same rationale as indicator matrices except for that the non-zero elements are a real number corresponding to the magnitude of the value. For instance, the concentration of a growth factor in the context of actions.

The indicator matrices can include, for example, a set of possible discrete actions and/or a set of possible cell states. An indicator matrix for a set of possible discrete actions can include, for instance, presence of a growth factor, where each element of the vector a_{α}(tₖ)=1 if action a is present at time point k and zero otherwise. An indicator matrix for a set of possible states can include, F=Iₙ=[aᵢⱼ], where i, j∈[[1, n]], [aᵢⱼ]=1 if i=j and zero otherwise.

Alternatively, the states and actions can be represented indirectly by, for example, encoding small molecule perturbation by, possibly weighted, indicator matrices of what known targets or pathways they affect; and/or encoding cell states by indicator matrices of transcription factors known to be expressed in the cell state of interest.

In various embodiments, the states and actions can be represented by a combination of direct and indirect representations. An advantage of indirect representations lies in that a model trained on one set of states and actions may transfer directly to another set of states and actions. The reason being that an indirect representation may be a general purpose one over many states and actions.

The solution can include alternative model formulations. In an embodiment, the cell differentiation process in Equation (4) can be viewed as a Markov process with a time-resolved transition matrix, where the transition matrix is learnt from data. Learning a complex relationship from data may be challenging if data availability is low, or the temporal dynamics of the process is complex. The solution, therefore, can include a modified formulation to facilitate modelling of temporal dynamics.

The solution can include delayered differential equations. For example, the solution can include a modified formulation such as a time-delay represented as:

$$\frac{d {x}_{i}}{d t} = {g}_{i , θ} ( t , {x}_{i} ( t ) , {x}_{t} ) \qquad ( 12 )$$

where xₜ={x(τ): τ≤t} represents the trajectory of past solutions. However, since g_{i,θ} is partially represented by the ML platform (for example, an ANN), it may be challenging to implement standard continuous delay. The solution can instead use a form using discrete delays as:

$$\frac{{dx}_{i}}{dt} = {g}_{i , θ} ( t , {x}_{i} ( t ) ) + {g}_{i , θ} ( t , {x}_{i} ( t - {τ}_{1} ) ) + … + {g}_{i , θ} ( t , {x}_{i} ( t - {τ}_{k} ) ) \qquad ( 13 )$$

Which naturally lends itself to algorithmic implementation. Delay differential equations can be used, for instance, in fully mechanistic modelling of hematopoietic stem cell dynamics modelling.

Since a cell state can be highly non-linear with respect to time, the solution can include dwell time assumptions into its system of equations. Rationale being that the temporal dynamics of cell state change itself depends on the temporal dynamics on many substates. For example, the production level of a single protein depends on the translation rate of the protein itself as well as protein levels of regulatory proteins. These rates in turn depend on the transcription rates of each gene encoding the protein and all regulatory steps in between. Although highly complex to implement in full, the solution can apply a linear chain trick (LCT, such as, for example, described in P. J. Hurtado and A. S. Kirosingh, “Generalizations of the ‘Linear Chain Trick’: incorporating more flexible dwell time distributions into mean field ODE models,” J. Math. Biol., vol. 79, no. 5, pp. 1831-1883 October 2019, doi: 10.1007/s00285-019-01412-w) to let g_{i,θ}(t, xᵢ(t)) depend on a chain of unobserved substates in addition to observed cell states.

A benefit of the instant technological solution lies in its use in control of stem cell differentiation. In various embodiments, the solution uses a predictive model to inform control and to make multiple predictions while varying the configuration of control parameters and measuring the effect on the prediction. Using this approach, it is possible to study the process in “what-if” scenarios. In the case of stem cell differentiation, the solution can be employed, for example, to determine the optimal time to add a molecule treatment and the optimal concentration of molecule treatment to increase the yield of a target cell type. For instance, based on the current composition of the cell culture, the solution can forecast how the culture will change under different configuration of time and concentration and choose, or facilitate choosing, the configuration that results in the highest predicted yield.

While endpoint prediction can provide an opportunity to inform decision making, predicting the endpoint yield allows optimization to increase endpoint yield, no more no less. Since the solution forecasts multiple timesteps, it allows decision making based on temporal evolution of the predicted parameter. For instance, by forecasting how the proportion of target cells increases over time, the solution can choose, or facilitate choosing, configurations that not only result in high yield, but also choose, or facilitate choosing, a configuration that reaches high yield fast. This temporal information brings clear economic advantages from a protocol optimization perspective. For example, in a nonlimiting implementation, an 8-day protocol can be shortened to an average of 7 days, or less, with sustained yield, thereby reducing the time during which resources are required by 12.5%, or more, with similar decrease in medium use due to fewer medium changes required.

As described in greater detail below, the solution includes a system, a computer-implemented method, and an apparatus for optimizing stem cell differentiation processes based on real-time monitoring using live cell measurement data. The solution does not only maximize yield, but it can optimize, or facilitate optimization of, multiple factors, such as, for example, speed of differentiation, viability, purity while minimizing line-to-line variability. This solution can predict and simulate what the future state of a cell culture will look like based on measurement data until the current time point under different potential future conditions. Based on this forecast, informed decisions can be made on how to adopt a protocol “on the fly”. If the solution simulates what will happen to a cell culture state under different simulated time points and concentrations of added growth factors, the timing and concentration can be adjusted to maximize a target objective. The solution can simulate different protocol conditions based on initial conditions alone, allowing full in-silico screening of protocol parameters without experiment.

In an embodiment, early termination of differentiation experiments can be implemented to save time and resources. Being able to forecast the future state of the cell culture, the solution can rapidly determine which experiments are unlikely to yield satisfactory results and terminate them early. This saves both experimental time as well as consumables.

In certain embodiments, the solution can simulate the effect of added growth factors or small compounds based on initial conditions and planned protocol. Due to being simulation-based, the solution can be used to rapidly screen many candidate protocols and run the most promising ones experimentally. Additionally, solution can be used to get an early readout of the protocol effect. During protocol development, the solution can be used to screen many potential conditions and how the influence differentiation results. Such screening experiments can entail many rounds of experiments where a set of culture conditions are evaluated at any given time. Using in-silico screening of a large number of candidate protocols, as well as simulations based on real-time monitoring to achieve an earlier readout of its impact, considerable resources can be saved. In at least one nonlimiting example, the solution can reduce screening periods from 9 days to 6 days, or less, thereby saving considerable amounts of time (time saved multiplied by the number of rounds required to finish) as well as consumables.

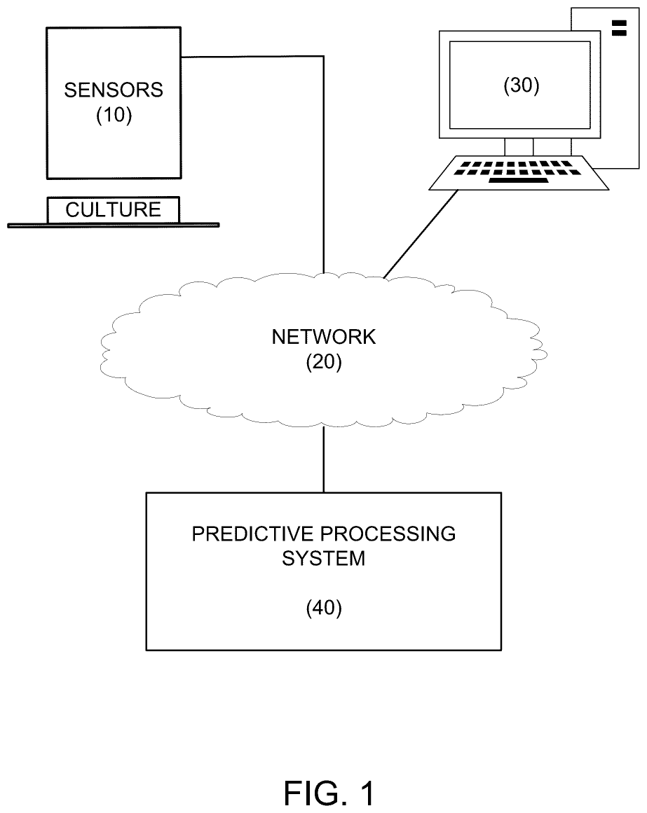

FIG. 1 shows a block diagram of a system for forecasting and/or simulating a cell culture state at one or more points in time, including predicting and/or simulating a yield of a target cell type i in the cell culture. The system includes one or more sensors 10, a network 20, a computing device 30, and a cell culture simulator 40. In various embodiments, the sensors 10 can include a variety of sensor devices, each configured to monitor and measure an attribute of a cell culture, including the attributes of individual cells, cell types, cell subtypes, and fates in the culture.

The sensor 10 can include one or more sensor devices configured to measure attributes such as inline or online process parameters, including at least one of pH, glucose, lactate, temperature, dissolved oxygen, spectroscopy (RAMAN, Near infrared, fourier transform infrared), conductivity for ionic strength and composition, optical density for biomass, capacitance for viable cell density, medium viscosity, redox potential for metabolic state and cell stress, mass spectrometry, and ultrasound-based measurement of fluid density.

The sensor 10 can include one or more sensor devices configured to measure attributes such as online or atline phenotypic parameters, including at least one of imaging and flow cytometry.

The sensor 10 can include at least one image pickup device, which can include, for example, a two-dimensional (2D) or three-dimensional digital image pickup device, such as, for example, a digital microscope camera, an electron microscope, or other high or ultrahigh resolution microscopic image pickup device (such as, for example, 1.5, 5, 10, 12, or 18 megapixels, or greater).

Each sensor 10 can be configured to monitor and measure an attribute of the cell culture and send the measurement data via a communication link to the computing device 30 or the cell culture simulator 40, including, for example, pH measurements, glucose measurements, lactate measurements, temperature measurements, pressure measurements, dissolved oxygen measurements, spectroscopy measurements, conductivity measurements, optical density measurements, capacitance measurements, medium viscosity measurements, redox potential measurements, mass spectrometry measurements, ultrasound-based measurements of fluid density, image data, and flow cytometry data. The measurement data can be sent directly, or via the network 20, to the computing device 30 or the cell culture simulator 40.

The computing device 30 can be configured to render (such as, for example, display) cell culture data, including measurement data representative of the current cell state, action(s), and a forecasted yield for each type of target cell type i at time t in the future. The information can be rendered by a human interface device, such as, for example, a graphic user interface (GUI) on a display device, an interactive voice response (IVR) unit and speaker, or other communication signal perceivable by a human user. The computing device 30 is further configured to receive annotations for each cell culture, including, for example, a label and description for each cell type, subtype, or fate, or other feature of the cell culture. The computing device 30 can be configured to communicate and interact with the sensors 10 and the cell culture simulator 40 via one or more communication links.

The cell culture simulator 40 is configured to receive real-time measurement data and store historical measurement data for a cell culture. The culture simulator 40 is configured to analyze, in real-time, the measurement data and historical data, including one or more measurements of a particular target cell culture, and forecast the future state of each cell type, subtype, or fate, in the cell culture undergoing differentiation based on the current state of the cell culture. The cell culture simulator 40 includes a machine learning (ML) platform and a mechanistic platform. The ML platform includes one or more ML models that are trained based on training datasets, and the mechanistic platform includes one or more mechanistic (M) models that are configured to interact with each other and simulate cell differentiation dynamics as a function of cell state, time, and actions, including as discussed above with respect to Equations (1) to (13).

The cell culture simulator 40 is configured to communicate with the sensors 10 and receive measurement data. The cell culture simulator 40 is also configured to communicate with the computing device 30 and receive a cell simulation request, including a cell target type and date/time information for a time point in the future for a particular cell culture. The cell culture simulator 40 is configured to monitor cell state and actions, including growth and changes in each cell, cell type, subtype, and fate, and compositions of cellular subpopulations in real-time (or near real-time). The cell culture simulator 40 can monitor the changes in each cell, cell type, subtype, and fate, including under varying actions, and predict the composition of cellular subpopulations in the target cell culture through time, including an absolute yield of various cell types, subtypes, and fates at a specified future time, by analyzing measurement data and historical data for the cell culture. The absolute yield can include the number of cells of a certain cell type, subtype, or fate, such as, for example, endoderm cells.

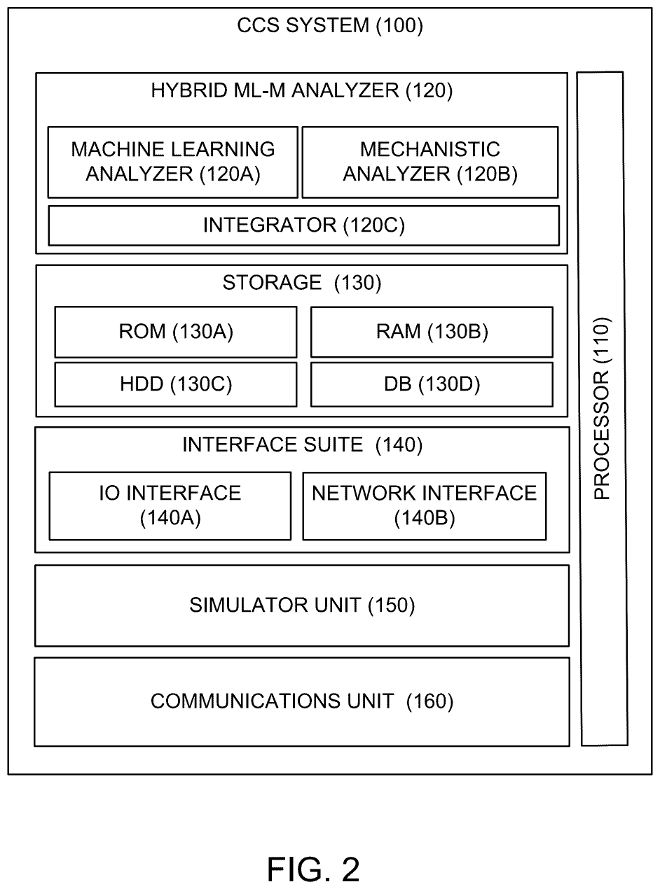

In at least one embodiment, the cell culture simulator 40 includes the Cell Culture Simulator (CCS) system 100 in FIG. 2. The CSS system 100 includes a processor 110, a hybrid machine learning—mechanistic (ML-M) analyzer 120, a storage 130, an interface suite 140, a simulator unit 150, and a communications unit 160. The CSS system 100 can include a bus (not shown) that can be connected to any or all of the components 110 to 160 by one or more communication links.

Any one or more of the components 110 to 160 can include a computing resource or a computing device. One or more of the components 120 to 160 can include a computing resource or a computing device that is separate from the processor 110, as seen in FIG. 2, or integrated with the processor 110. In certain embodiments, one or more of the components 120 and 140 to 160 can include a computer resource that can be executed on the processor 110 as one or more processes. The computer resources can be contained in the storage 130.

The bus can include any of several types of bus structures that can further interconnect to a memory bus (with or without a memory controller), a peripheral bus, and a local bus using any of a variety of commercially available bus architectures.

The processor 110 can include one or more processors, such as, for example, ab application specific integrated circuit (ASIC) and any of various commercially available processors, including for example, a central processing unit (CPU), a graphic processing unit (GPU), a general-purpose GPU (GPGPU), a dedicated neural processing unit (NPU), a tensor processing unit (TPU), a field programmable gate array (FGPA), an application-specific integrated circuit (ASIC), a system-on-a-chip (SOC), a single-board computer (SBC), a manycore processor, multiple microprocessors, or any other computing device architecture. The processor 110 can be arranged to interact with any of the components 120 to 160 to carry out or facilitate the processes included, described or contemplated by this disclosure. The processor 110 can be arranged to run one or more machine or deep learning systems.

The processor 110 can be arranged to run an operating system (OS), which can include an operating system (OS) kernel that can control all operations on the CCS system 100. The OS kernel can include, for example, a monolithic kernel or a microkernel. The OS kernel can be arranged to execute on the processor 110 and have control over operations in the processor 110.

The OS or OS kernel can be contained in the storage 130 and executed by the processor 110. The OS or OS kernel can be cached in the storage 130, such as, for example, in a random-access memory (RAM) 130B. The OS kernel can represent the highest level of privilege on the OS or the processor 110. The OS can include a driver for each hardware device with which the processor 110 might interact, including, for example, one or more receivers, transmitters, or transceivers in the communications unit 160. The OS kernel can be arranged to allocate resources or services to and enable computing resources or processes to share or exchange information, protect the resources or services of each computing resource or process from other computing resources or processes, or enable synchronization amongst the computing resources or processes.

The OS kernel can, when a process is triggered, initiate and carry out the process for that computer resource, including allocating resources for the process, such as, for example, hard disk space, memory space, processing time or space, or other services on one or more hardware devices in the CCS system 100. The OS kernel can carry out the process by allocating memory space and processing resources to the process, loading the corresponding computing resource (or portion of a computing resource) into the allocated memory space, executing instructions of the computing resource on the OS kernel, or interfacing the process to one or more computer resources or processes.

The OS kernel can be arranged to facilitate interactions between the computing resources or processes. The processor 110, which runs the OS, can be arranged to arbitrate access to services and resources by the processes, including, for example, running time on the processor 110. The OS kernel can be arranged to take responsibility for deciding at any time which of one or more processes should be allocated to any of the resources.

The hybrid ML-M analyzer 120 can include a plurality of computer resources and/or computing devices. The ML-M analyzer 120 includes a machine learning analyzer 120A, a mechanistic analyzer 120B, and an integrator 120C, any one or more of which can include a computer resource and/or a computing device. The hybrid ML-M analyzer 120 forecasts and simulates the population differentiation dynamics of a cell culture using the machine learning and mechanistic methodologies discussed above, while reducing the amount of data necessary, for example, to train the one or more machine learning (ML) models in the machine learning platform. The ML-M analyzer 120 includes a plurality of models (including at least one ML model and at least one M-model) configured to describe how a composition of discrete states evolve over time in a cell culture of mixed states. The cell culture can include a stem cell culture undergoing differentiation, wherein the discrete states include different cell types, subtypes, or fates. The cell subtypes may be the starting and target cell type or fate of differentiation, as well as any subtypes the cells may differentiate into during differentiation. The discrete states may also be quantified as discretized expression levels of one or more biomarkers, for instance gene expression of a transcription factor.

The ML analyzer 120A can include one or more computing resources, each arranged to run on the processor 110, or it can include one or more computing devices, each arranged to interact with the mechanistic analyzer 120B and integrator 120C, as well as one or more of the components 130-160.

In various embodiments, the ML analyzer 120A includes a supervised ML platform, an unsupervised ML platform, or both supervised and unsupervised ML platforms. The machine learning platform includes one or more machine learning models built to predict one or more parameters for a mechanistic (M) model in the mechanistic analyzer 120B, which then imposes constraints or restrictions (as discussed above) to reduce the amount of data necessary to train or tune the ML model(s).

The one or more ML models in the ML analyzer 120A are built to forecast and simulate the future state of a cell culture undergoing differentiation given initial conditions, real-time measurements, and past and future perturbations to the cell culture. The one or more ML models can forecast and simulate, for example, the rate of change of cellular subpopulations in the cell culture, as well as the number of cells of each state, cell type, subtype, and fate at a particular time point in the future. The ML model(s) can forecast and simulate the time-trajectory of how cellular subpopulations change to either predict future timepoints or interpolate between discrete measurements.

The mechanistic analyzer 120B can include one or more computer resources and/or computing devices, each configured to carry out the mechanistic Equations set forth above. The mechanistic analyzer 120B includes one or more models built to perform the mechanistic calculations discussed above, thereby reducing the amount of data necessary to train or tune the one or more ML models in the ML analyzer 120A.

The integrator 120C can include one or more computer resources and/or computing devices, each configured to carry out the integration calculations set forth above. In certain embodiments, the integrator 120C can be incorporated in the mechanistic analyzer 120B.

The CCS system 100 can include a non-transitory computer-readable storage medium that can hold executable or interpretable computer resources, including computer program code or instructions that, when executed by the processor 110, cause the steps, processes or methods in this disclosure to be carried out, including training, tuning and operation of machine learning platform in the machine learning (ML) analyzer 120A, and operation of the mechanistic analyzer 120B, integrator 120C, and simulator unit 150. The computer-readable storage medium can be contained in the storage 130 or an external storage device (not shown).

The storage 130 can include a read-only memory (ROM) 130A, a random-access memory (RAM) 130B, a hard disk drive (HDD) 130C, and a database (DB) 130D. The storage 130 can provide nonvolatile storage of data, data structures, and computer-executable instructions, and can accommodate the storage of any data in a suitable digital format.

The storage 130 can include the non-transitory computer-readable medium that can hold the computer resources (including code or instructions) that can be executed (run) or interpreted by the operating system on the processor 110. The computer-readable medium can be contained in the HDD 130C.

A basic input-output system (BIOS) can be stored in the non-volatile memory in the ROM 130A, which can include, for example, an erasable programmable read-only memory (EPROM), or an electrically erasable programmable read-only memory (EEPROM). The BIOS can contain the basic routines that help to transfer information between any one or more of the components 110 to 160 in the CCS system 100, such as during start-up.

The RAM 130B can include a dynamic random-access memory (DRA M), a synchronous dynamic random-access memory (SD RAM), a static random-access memory (SRAM), a non-volatile random-access memory (NV RAM), or another high-speed RAM for caching data.

The HDD 130C can include, for example, an enhanced integrated drive electronics (EIDE) drive, a serial advanced technology attachments (SATA) drive, or any suitable hard disk drive for use with big data. The HDD can be configured for external use in a suitable chassis (not shown). The HDD can be arranged to connect to the bus B via a hard disk drive interface (not shown). In various nonlimiting embodiments, the HDD 130C can include the machine learning (ML) platform and/or the mechanistic platform.

The DB 130D can be arranged to be accessed by any one or more of the components in the system 100. The DB 130D can be arranged to receive a request and, in response, retrieve specific data, data records or portions of data records based on the request. A data record can include, for example, a file or a log. The DB 130D can include a database management system (DBMS) that can interact with the components 110 to 160. The DBMS can include, for example, SQL, NoSQL, MySQL, Oracle, Postgress, Access, or Unix. The DB 130D can include a relational database.

The DB 130D can be arranged to contain machine learning training datasets, testing datasets, and historical data. The DB 130D can contain information related to each cell culture, cell type, subtype, and fate.

Any number of computer resources can be stored in the storage 130, including, for example, a program module, an operating system (not shown), one or more application programs (not shown), or program data (not shown). Any (or all) of the operating system, application programs, program modules, and program data can be cached in the RAM as executable sections of computer code.

The network suite 140 includes an input/output (IO) interface 140A and a network interface 140B. The IO interface 140A can receive instructions or data from an operator via a human interface device (not shown), such as, for example, a keyboard (not shown), a mouse (not shown), a pointer (not shown), a stylus (not shown), a microphone (not shown), an interactive voice response (IVR) unit (not shown), a speaker (not shown), or a display device (not shown). The received instructions and data can be forwarded from the IO interface 140A as signals via one or more communication links to any component in the system 100.

The network interface 140B can connect to the network 20 (shown in FIG. 1). The network interface 140B can be arranged to communicate with any number of devices (such as, for example, the sensors 10 or the computing device 30, shown in FIG. 1), either directly or via the network 20 over one or more communication links. The network interface 140B can include a wired or wireless communication network interface (not shown) or a wired or wireless modem (not shown). When used in a local area network (LAN), the network interface 140B can connect to the LAN network through the communication network interface; and, when used in a wide area network (WAN), it can connect to the WAN network through the modem. The modem (not shown) can be connected to the system bus via, for example, a serial port interface (not shown). The network interface 140B can be arranged to interact with the communications unit 160, or it can include a receiver (not shown), transmitter (not shown) or transceiver (not shown).

The simulator unit 150 can include one or more computer resources and/or computing devices, each configured to generate one or more multimedia signals that can be rendered by a human interface device, such as, for example, the display device of the computing device 30 (shown in FIG. 1). The multimedia signals can include audio and video signals that can be, for example, rendered by the human interface device, for example, as a displayed image or reproduced sound. The multimedia signals include yield and time information, including a forecasted yield for a target cell type, subtype, or fate, and the time point of the forecasted yield. The multimedia signals can include current cell state information, measurement data, and actions applied to, or performed on the cell culture.

The communications unit 160 can include one or more transmitters, receivers, or transceivers. The communications 160 can be configured to communicate with sensor devices and computing devices, including the sensors 10 and computing device 30 (shown in FIG. 1).

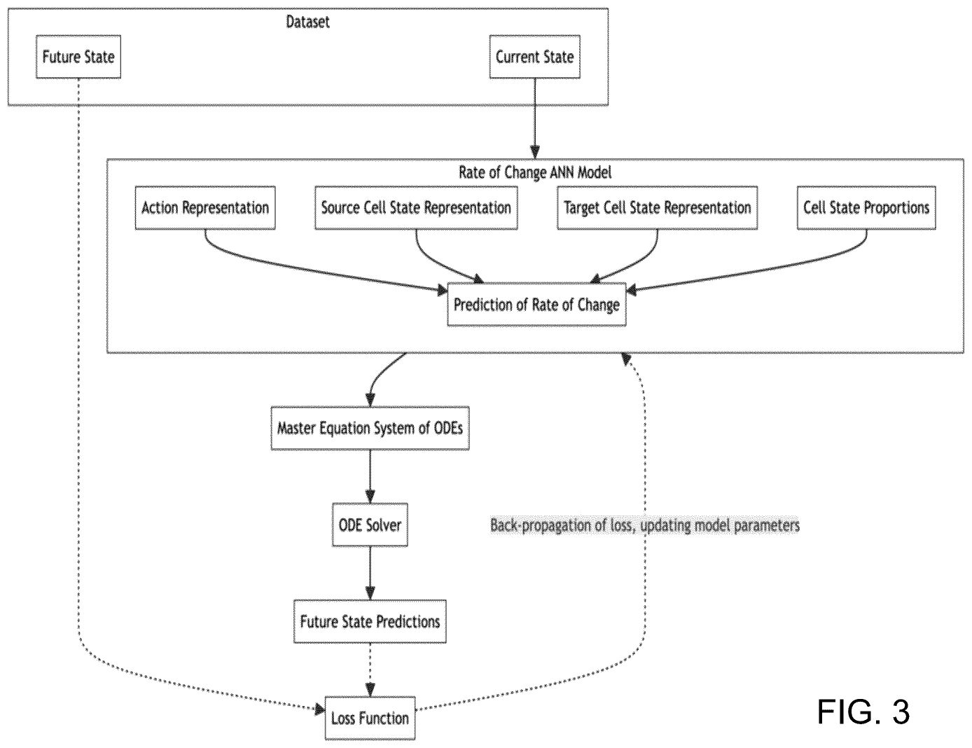

FIG. 3 shows a block diagram of a nonlimiting example of the hybrid model implementation by the CCS system 100 (shown in FIG. 2). Referring to FIGS. 2 and 3 contemporaneously, the CCS system 100 can receive measurement data for the current state of a cell culture from the sensors 10 (shown in FIG. 1) and forecast a future state of the cell culture. The future state and current state datasets can be stored in the storage 130.

Based on the measurement data for (and representative of) the current state of the cell culture, the ML model (for example, ANN model) in the ML analyzer 120A can forecast a rate of change for each cell, cell type, subtype, or fate based on action representation, cell state representation, target cell state representation and cell state proportions, as discussed earlier. The output from the ML model is fed to the mechanistic model in the M-analyzer 120B and/or integrator 120C, wherein a master equation system of ODEs, an ODE solver, and future state predictions are carried out. The M-analyzer 120B/integrator 120C performs a loss function to provide back-propagation of loss to the ML model to update and tune the model's parameters in the ML analyzer 120A.

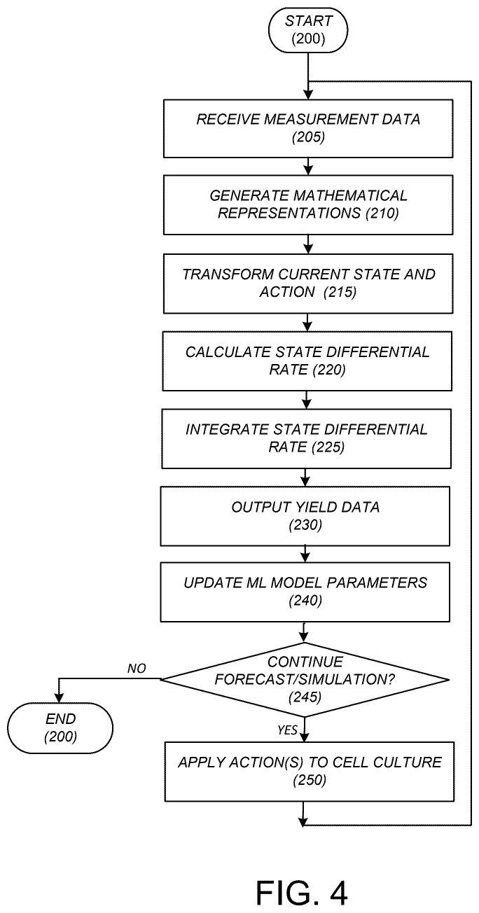

FIG. 4 shows an embodiment of a process for predicting and simulating cell differentiation dynamics for a cell culture undergoing differentiation.

Referring to FIGS. 1, 2 and 4 contemporaneously, the CSC system 100 can receive a request from the computing device 30 for a yield of a target cell type i at a specific time τ in the future for cell culture undergoing differentiation. After (or independent of) receiving the request, the CSC system 100 receives one or more measurements (for example, from the sensors 10) of the cell culture containing n discrete states of cells undergoing cell differentiation at time t, where n is a positive integer (Step 205). The received one or more measurements of the cell culture are fed to the ML platform (for example, ML analyzer 120A). Based on the measurements, the ML platform generates a mathematical representation for each of the n discrete states based on the measurements (Step 210). The mathematical representation includes a current state x(t) and an action α(t) for each of the n discrete states at time t, including a current state xᵢ(t) and an action αᵢ(t) for the target cell type i.

The ML platform then transforms, for each of the n discrete states, the current state x(t) and the action α(t) to a state change rate r_{θ}, where θ is a parametric weight value mapped by the machine learning platform to the current state x(t) and the action α(t) for each of the n discrete states (Step 215). The state change rate r_{θ} can then be input to the mechanistic (M−) model, which calculates a state differential rate dx/dt for each of the n discrete states, including a state differential rate dxᵢ/dt for the target cell type i, based on the state change rate r_{θ} (Step 220). The state differential rate dx/dt for each of the n discrete states is fed to the integrator 120C, which integrates the state differential rate dx/dt for each of the n discrete states from an initial time t₀ to the time τ to calculate the yield of the target cell type i at time τ (Step 225).

The calculated yield is output to, for example, the simulator 150 and/or the communications unit 160 (Step 230). The simulator 150 can generate a multimedia signal containing the calculated yield and output, or send via the communications unit 160, the multimedia signal to a human interface display (for example, the display device on the computing device 30) to display the yield and any associated data, such as, for example, measurement data, cell state data, and action data.

A loss function can be applied to the output from the integrator 120C to provide back-propagation of loss to update the model parameters of the ML model (for example, in the ML analyzer 120A) (Step 240).

A determination can be made whether additional simulation is requested (Step 245), including, for example, to test different actions on the cell culture and forecast the effects of such actions on the yield of a target cell, cell type, subtype, or fate. If additional forecasting is requested (Y ES at Step 245), then one or more additional actions can be applied (or simulated) to the cell culture (Step 250), and the process 200 repeated, otherwise (NO at Step 245) the process ends.

The terms “a,” “an,” and “the,” as used in this disclosure, means “one or more,” unless expressly specified otherwise.

The term “action” as used herein, can for example, refer to maintaining the cell culture state, modulating the cell culture state, ending the cell culture, requesting further measurements, and/or requesting complementary measurements. By way of an example, maintaining the cell culture state can include maintaining the present cell culture conditions without changing the media. By way of an example, modulating the cell culture state can include adding or removing specific growth factors, renewing or refreshing the cell culture media, adding or removing serum in the cell culture media, and/or generally changing the current cell culture state. By way of an example, ending the cell culture can comprise stopping the cell culture from proceeding. By way of an example, requesting further measurements can include requesting additional inline process parameters or online process parameters such as, e.g., a pH measurement, a temperature measurement, a glucose measurement, a lactate measurement, a dissolved oxygen measurement, a spectroscopy measurement, a conductivity measurement, an optical density measurement, a capacitance measurement, a medium viscosity measurement, a redox potential measurement, a mass spectrometry measurement, and/or an ultrasound-based measurement of fluid density. By way of another example, requesting further measurements can include requesting additional online phenotypic measurements or atline phenotypic measurements such as, e.g., one or more images or a flow cytometry analysis. By way of another example, requesting complementary measurements can comprises requesting inline or online process parameters or online or atline phenotypic measurements that complement a measurement already taken.

The term “backbone,” as used in this disclosure, means a transmission medium that interconnects one or more computing devices or communicating devices to provide a path that conveys data signals and instruction signals between the one or more computing devices or communicating devices. The backbone can include a bus or a network. The backbone can include an ethernet TCP/IP. The backbone can include a distributed backbone, a collapsed backbone, a parallel backbone or a serial backbone.

The term “bus,” as used in this disclosure, means any of several types of bus structures that can further interconnect to a memory bus (with or without a memory controller), a peripheral bus, or a local bus using any of a variety of commercially available bus architectures. The term “bus” can include a backbone.

The term “communication link,” as used in this disclosure, means a wired or wireless medium that conveys data or information between at least two points. The wired or wireless medium can include, for example, a metallic conductor link, a radio frequency (RF) communication link, an Infrared (IR) communication link, or an optical communication link. The RF communication link can include, for example, WiFi, WiMAX, IEEE 802.11, DECT, OG, 1G, 2G, 3G, 4G, or 5G cellular standards, or Bluetooth. A communication link can include, for example, an RS-232, RS-422, RS-485, or any other suitable serial interface.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The terms “computer,” “computing device,” or “processor,” as used in this disclosure, means any machine, device, circuit, component, or module, or any system of machines, devices, circuits, components, or modules that are capable of manipulating data according to one or more instructions. The terms “computer,” “computing device” or “processor” can include, for example, without limitation, a communicating device, a computer resource, a processor, a microprocessor (μC), a central processing unit (CPU), a graphic processing unit (GPU), an application specific integrated circuit (ASIC), a general purpose computer, a super computer, a personal computer, a laptop computer, a palmtop computer, a notebook computer, a desktop computer, a workstation computer, a server, a server farm, a computer cloud, or an array or system of processors, μCs, CPUs, GPUs, ASICs, general purpose computers, super computers, personal computers, laptop computers, palmtop computers, notebook computers, desktop computers, workstation computers, or servers.

The terms “computing resource” or “computer resource,” as used in this disclosure, means software, a software application, a web application, a web page, a computer application, a computer program, computer code, machine executable instructions, firmware, or a process that can be arranged to execute on a computing device as one or more processes.

The term “computer-readable medium,” as used in this disclosure, means any non-transitory storage medium that participates in providing data (for example, instructions) that can be read by a computer. Such a medium can take many forms, including non-volatile media and volatile media. Non-volatile media can include, for example, optical or magnetic disks and other persistent memory. Volatile media can include dynamic random-access memory (DRA M). Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, a carrier wave as described hereinafter, or any other medium from which a computer can read. The computer-readable medium can include a “cloud,” which can include a distribution of files across multiple (e.g., thousands of) memory caches on multiple (e.g., thousands of) computers.

Various forms of computer readable media can be involved in carrying sequences of instructions to a computer. For example, sequences of instruction (i) can be delivered from a RAM to a processor, (ii) can be carried over a wireless transmission medium, or (iii) can be formatted according to numerous formats, standards or protocols, including, for example, WiFi, WiMAX, IEEE 802.11, DECT, OG, 1G, 2G, 3G, 4G, 5G, or 6G cellular standards, or Bluetooth.

The term “current state of a cell culture,” as used herein, refers to the state of the cell culture at the time a request for a yield of a target cell type is received by the one or more processors. The current state of a cell culture can, for example, refer to the instant state of the cell culture at the time the one or more measurements of the cell culture are being made, i.e., the inline process parameter, the online process parameter, the online phenotypic measurement, and/or the atline phenotypic measurement. The inline process parameter or online process parameter can, for example, include a pH measurement, a temperature measurement, a glucose measurement, a lactate measurement, a dissolved oxygen measurement, a spectroscopy measurement, a conductivity measurement, an optical density measurement, a capacitance measurement, a medium viscosity measurement, a redox potential measurement, a mass spectrometry measurement, and/or an ultrasound-based measurement of fluid density. The online phenotypic measurement or the atline phenotypic measurement can, for example, include one or more images of the cell culture or a flow cytometry analysis of the cell culture.

The term “database,” as used in this disclosure, means any combination of software or hardware, including at least one computing resource or at least one computer. The database can include a structured collection of records or data organized according to a database model, such as, for example, but not limited to at least one of a relational model, a hierarchical model, or a network model. The database can include a database management system application (DBMS). The at least one application may include, but is not limited to, a computing resource such as, for example, an application program that can accept connections to service requests from communicating devices by sending back responses to the devices. The database can be configured to run the at least one computing resource, often under heavy workloads, unattended, for extended periods of time with minimal or no human direction.

The term “future state of a cell culture,” as used herein, refers to the state of the cell culture at any time point in the future. By way of an example, the future state of a cell culture can be the state of the cell culture at the end of the experiment (i.e., at the end of a stem cell differentiation process). Alternatively, the future state of a cell culture can be at least 1 day, at least 2 days, at least 3 days, at least 4 days, at least 5 days, at least 6 days, at least 7 days, at least 8 days, at least 9 days, or at least 10 days from the current day that the request for a yield of a target cell type is provided to the one or more processors. Alternatively, the future state of a cell culture can be at least 1 hour, at least 2 hours, at least 3 hours, at least 4 hours, at least 5 hours, at least 6 hours, at least 7 hours, at least 8 hours, at least 9 hours, or at least 10 hours from the current hour that the request for a yield of a target cell type is provided to the one or more processors. The future state of the cell culture can refer to the yield of a target cell type of the cell culture, i.e., the amount of the target cell type that arises during the stem cell differentiation process; the growth of the target cell type during the stem cell differentiation process; and/or the specific composition of the subpopulations of cells in the stem cell culture during the differentiation process or at the conclusion of the differentiation process.

The terms “including,” “comprising” and their variations, as used in this disclosure, mean “including, but not limited to,” unless expressly specified otherwise.

The term “network,” as used in this disclosure means, but is not limited to, for example, at least one of a personal area network (PAN), a local area network (LAN), a wireless local area network (W LAN), a campus area network (CAN), a metropolitan area network (MAN), a wide area network (WAN), a metropolitan area network (MAN), a wide area network (WAN), a global area network (GAN), a broadband area network (BAN), a cellular network, a storage-area network (SAN), a system-area network, a passive optical local area network (POLAN), an enterprise private network (EPN), a virtual private network (VPN), the Internet, or the like, or any combination of the foregoing, any of which can be configured to communicate data via a wireless and/or a wired communication medium. These networks can run a variety of protocols, including, but not limited to, for example, Ethernet, IP, IPX, TCP, UDP, SPX, IP, IRC, HTTP, FTP, Telnet, SMTP, DNS, ARP, ICMP.

The term “server,” as used in this disclosure, means any combination of software or hardware, including at least one computing resource or at least one computer to perform services for connected communicating devices as part of a client-server architecture. The at least one server application can include, but is not limited to, a computing resource such as, for example, an application program that can accept connections to service requests from communicating devices by sending back responses to the devices. The server can be configured to run the at least one computing resource, often under heavy workloads, unattended, for extended periods of time with minimal or no human direction. The server can include a plurality of computers configured, with the at least one computing resource being divided among the computers depending upon the workload. For example, under light loading, the at least one computing resource can run on a single computer. However, under heavy loading, multiple computers can be required to run the at least one computing resource. The server, or any if its computers, can also be used as a workstation.

The terms “send,” “sent,” “transmission,” or “transmit,” as used in this disclosure, means the conveyance of data, data packets, computer instructions, or any other digital or analog information via electricity, acoustic waves, light waves or other electromagnetic emissions, such as those generated with communications in the radio frequency (RF) or infrared (IR) spectra. Transmission media for such transmissions can include coaxial cables, copper wire and fiber optics, including the wires that comprise a system bus coupled to the processor.

The term “specific cell type or cell subtypes” as used herein, can, for example, refer to the starting cell culture, i.e., the starting type of stem cell, and each specific type and/or stage of cell produced during the differentiation process, i.e., a cell type for creating a mesoderm (e.g., a skeletal muscle cell, a cardiac muscle cell (i.e., a cardiomyocyte), a kidney cell, a red blood cell, or a smooth muscle cell), an endoderm (e.g., a lung cell, a thyroid cell, or a pancreatic cell), and/or an ectoderm (e.g., a skin cell, a neural cell, or a pigment cell).

The term “specific fate of the cell” as used herein, can, for example, include (1) whether the cells are alive or dead; (2) whether the cells are capable of fully undergoing the stem cell differentiation process; (3) whether the cells have senesced, i.e., reached the endpoint of the differentiation process or reached the end of the growth phase; (4) whether the cells have produced the particular factors needed for the differentiation process; undergone the specific morphological changes needed for the differentiation process, which could include axon formation, dendrite formation, and/or dendritic spine formation; or undergone an activation needed for the differentiation process, which could include T-cell activation and/or B-cell activation; and/or (5) the specific stage of the cell cycle in which the cells currently reside.

The term “yield of a target cell type” as used in this disclosure, means the amount of number of cells of the target cell type that arise during the stem cell differentiation process. By way of an example a stem cell can be differentiated into three germ layers, the mesoderm, the endoderm, and/or the ectoderm. Yield can refer to the amount of endoderm cells or any progenitor cell thereof produced at any point during the stem cell differentiation process. “Yield of a target cell type” can, for example, also refer to the growth of the target cell type during the stem cell differentiation process, i.e., the division rate of each target cell type. “Yield of a target cell type” can, for example, also refer to the specific composition of the subpopulations of cells in the cell culture, i.e., the composition of the populations of stem cells, differentiated stem cells, and any progenitor cells produced during the differentiation process that have not fully differentiated to the final target cell type. Yield can, for example, refer to “absolute yield” or “relative yield.” As used herein, “absolute yield” refers to the number of cells of the target cell type. As used herein, “relative yield” refers to the percentage of target cell type out of all cells in the culture.

Devices that are in communication with each other need not be in continuous communication with each other unless expressly specified otherwise. In addition, devices that are in communication with each other may communicate directly or indirectly through one or more intermediaries.

Although process steps, method steps, or algorithms may be described in a sequential or a parallel order, such processes, methods and algorithms may be configured to work in alternate orders. In other words, any sequence or order of steps that may be described in a sequential order does not necessarily indicate a requirement that the steps be performed in that order; some steps may be performed simultaneously. Similarly, if a sequence or order of steps is described in a parallel (or simultaneous) order, such steps can be performed in a sequential order. The steps of the processes, methods or algorithms described in this specification may be performed in any order practical.

When a single device or article is described, it will be readily apparent that more than one device or article may be used in place of a single device or article. Similarly, where more than one device or article is described, it will be readily apparent that a single device or article may be used in place of the more than one device or article. The functionality or the features of a device may be alternatively embodied by one or more other devices which are not explicitly described as having such functionality or features.

The subject matter described above is provided by way of illustration only and should not be construed as limiting. Various modifications and changes can be made to the subject matter described herein without following the example embodiments and applications illustrated and described, and without departing from the true spirit and scope of the invention encompassed by the present disclosure, which is defined by the set of recitations in the following claims and by structures and functions or steps which are equivalent to these recitations.

### EXAMPLES

### Example 1

Stem cell culturing: Human Pluripotent cells were maintained under essential E8 (A 1517001, Thermo Fisher, Waltham, MA) medium on Vitronectin (AF-140-09, Peprotech)-coated plates. The medium was changed daily.

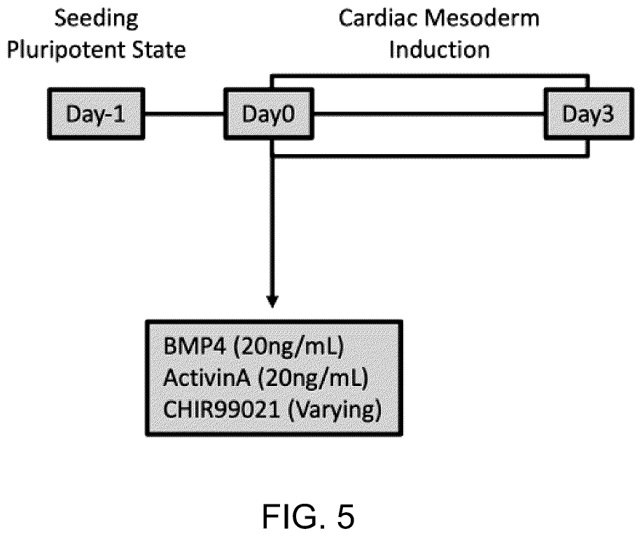

Stem cell differentiation: To differentiate cells into cardiac mesoderm cells, cardiomyocytes and/or endothelial intermediate, protocol described in Giacomelli et al., [1] was adapted. A schema of the experimental plan is shown in FIG. 5.

Briefly, human pluripotent cells (hPCs) were seeded, as a monolayer culture, into 96-well imaging plate in BPLE basal media for twenty-four hours [2,3], with the bovine serum albumin (BSA) polyvinylalcohol essential lipids (BPLE) basal media being supplemented with 20 ng/ml of ActinvinA (Peprotech, 120-14E-2 μG), 20 ng/ml of BMP4 (Peprotech, 120-05ET-2 μG) and varying concentrations of CHIR99201 (Selleckchem, S1263). CHIR99021 was removed randomly after either 24 or 48 hours of differentiation, to determine the effect of CHIR99021 on mesoderm differentiation rates.

### Ground Truth Generation:

To prove correct cardiac mesoderm induction and investigate the effect of varying concentrations of CHIR99201, every twelve hours of differentiation, cells were stained for the expression of the Mesoderm marker Brachyury. Briefly, cells were fixed in 4% PFA for 10 minutes, subsequently blocked in PBS 0.01% Triton and 3% BSA for 1 hour at room temperature and incubated with the primary antibody (1:200) overnight. The day after, the primary antibody was washed out, and the cells were incubated with the fluorescence-conjugated secondary antibody (1:1000) for 1 hour at room temperature. DAPI solution was used to detect nuclei.

Ground truth for fate forecasting was inferred by using Cellpose [4] to segment individual cell nuclei on the DAPI channel. A Multi-Otsu threshold was carried out on the Brachury channel to separate the images into foreground and background. The total number of nucleii detected was recorded and the two sets of masks were correlated to determine the proportion of cells that were positive for the immunofluorescence marker. This ratio was utilized as yield of target cell type for downstream analysis and modelling purposes.

### Image Acquisition:

Cells were imaged for the entire duration of the differentiation experiment from fluorescent channel(s) (according to experimental needs) and/or Phase contrast. Acquisition time varied across experiments.

### FateSim Model:

The FateSim model consisted of two parts: a neural network model that predicted the transition rates between different cell fates, and a yield simulator that used the predicted rates to update cell fate yields. The model predicted the yields of two fates: the unknown fate (corresponding to undifferentiated cells) and the mesoderm fate (corresponding to the target cell type). The model took as an input the concentration of the drug CHIR99021. The model that predicted fate transition rates was a feedforward network consisting of 4 linear layers, which took as an input the source/target cell fates and current drug concentration and then outputted a predicted transition rate for each source/target pair. The predicted fate transition rates were passed to a Master Equation yield simulator that predicted the inflow and outflow of fates utilizing an ODE solver. The yield simulator used the following model hyperparameters: r\hat{ }θ_{δ} is disabled and r\hat{ }θ_{div} is active.

### Training:

The model was trained for 200 epochs with a learning rate of 0.001. The training data was based on the extracted ground truth of a 3-day cardiac mesoderm induction differentiation experiment. The total number of detected cells along with the number of Brachury marker positive cells were used to provide training targets for the predicted yields.

### Results

Below are the results of the FateSim prediction model on two example hold-out wells that were not utilized as part of the training.

For one well, CHIR99021 was applied for 24 hours at a concentration of 0.93 μM, prior to being removed and replaced with media not including CHIR99021.

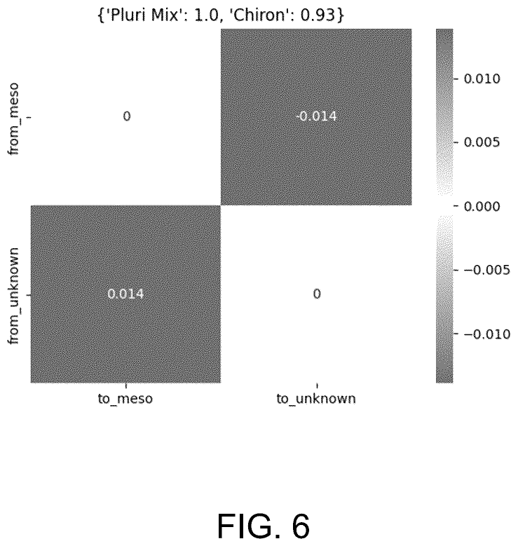

FateSim correctly predicts that reduced concentration of CHIR99021 (0.93 μM) corresponds to a diminished differentiation rate (1.4%/h) (FIG. 6) from pluripotency to mesodermal lineage.

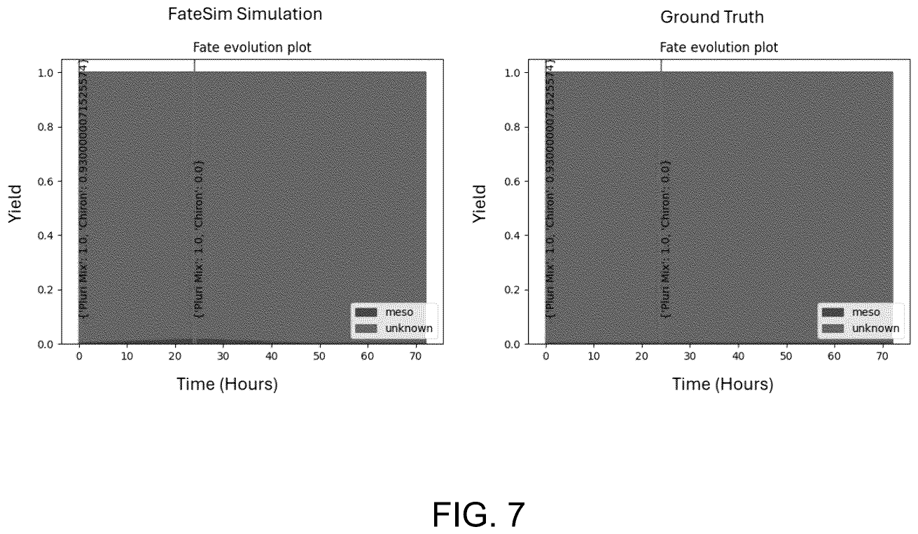

This diminished differentiation rate is predicted to be insufficient to drive the transition of pluripotent cells towards the mesodermal lineage fate. (FIG. 7). This was corroborated with the observed ground truth data, which showed a very low proportion of the cells acquiring the mesodermal cell fate.

For the other well, CHIR99021 was applied at a higher concentration of 1.93 μM and removed after 48 hours of differentiation.

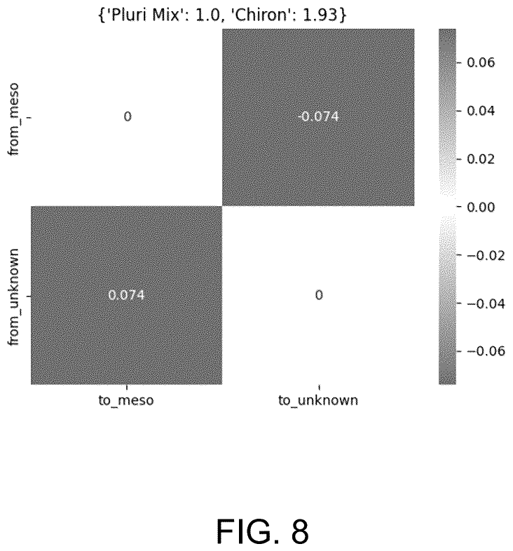

FateSim correctly predicted that increased concentration of CHIR99021 (1.93 μM) corresponded to an increased differentiation rate (7.4%/h) from pluripotency to mesodermal lineage (FIG. 8).

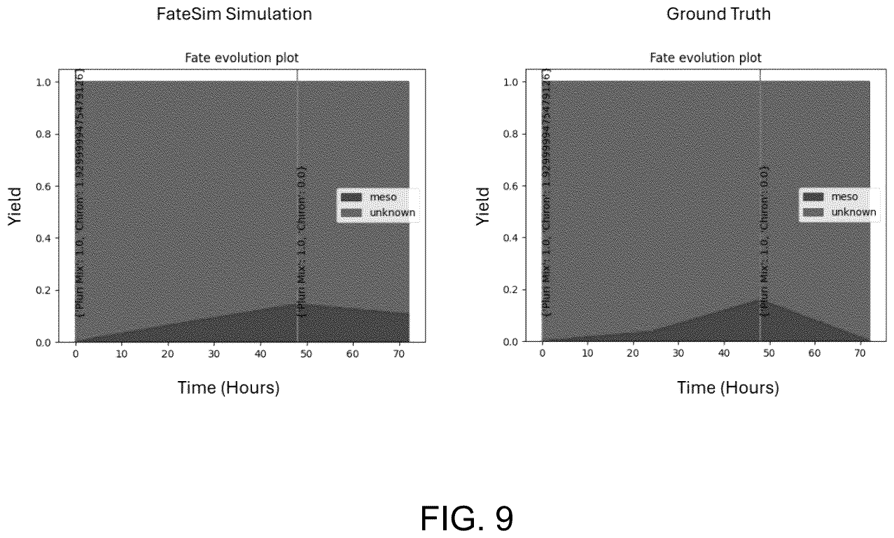

FateSim correctly predicted that there was an increase in Mesoderm cell fate up until the 48 hour mark, after which CHIR99021 was removed. This removal led to a decrease in the amount of Brachury positive cells that were predicted by the model, which was corroborated by the observed ground truth results (FIG. 9).

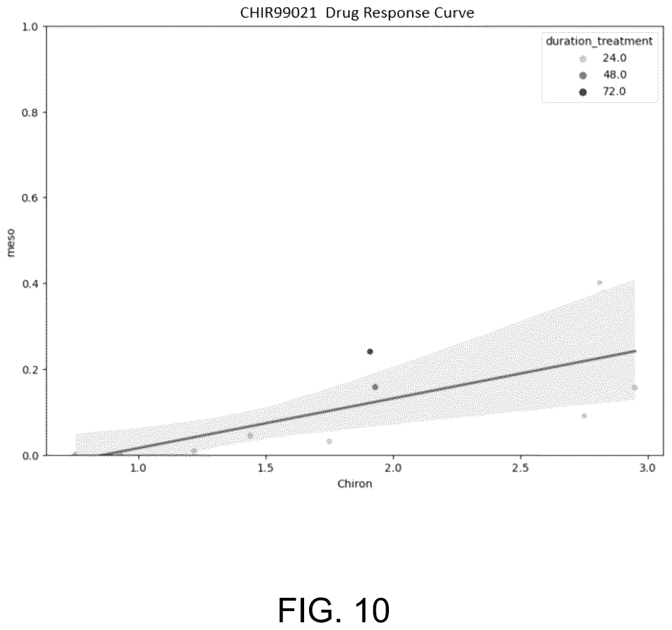

The FateSim model was able to predict a drug response curve to CHIR99021 concentrations and was able to predict the yield of mesoderm fate utilizing this curve (FIG. 10).

### REFERENCES

[1] Giacomelli E., et al. “Three-dimensional cardiac microtissues composed of cardiomyocytes and endothelial cells co-differentiated from human pluripotent stem cells”. Development, 2016, doi: 10.1242/dev.143438.
[2] Ng, E. S., et al. “A protocol describing the use of a recombinant protein-based, animal product-free medium (APEL) for human embryonic stem cell differentiation as spin embryoid bodies”. Nature Protocols, 2008, doi: 10.1038/nprot.2008.42.
[3] Campostrini, G., et al. “Generation, functional analysis and applications of isogenic three-dimensional self-aggregating cardiac microtissues from human pluripotent stem cells”. Nature Protocols, 2012, doi: 10.1038/s41596-021-00497-2.
[4] C. Stringer, T. Wang, M. Michaelos, and M. Pachitariu, “Cellpose: a generalist algorithm for cellular segmentation,” Nat. Methods, vol. 18, no. 1, Art. no. 1, January 2021, doi: 10.1038/s41592-020-01018-x.

1. A computer-implemented method for optimizing a stem cell differentiation based on real-time monitoring of the stem cell culture undergoing differentiation, the method comprising:
(a) receiving, by a processor, a request for a yield of a target cell type i at a specific time τ in the future;
(b) receiving, by the processor, one or more measurements of a cell culture containing n discrete states of cells undergoing cell differentiation at time t, where n is a positive integer;
(c) providing, by the processor, the one or more measurements of the cell culture to a machine learning platform;
(d) generating, by the machine learning platform, a mathematical representation for each of the n discrete states based on the one or more measurements, the mathematical representation including a current state x(t) and an action α(t) for each of the n discrete states at time t, including a current state xᵢ(t) and an action αᵢ(t) for the target cell type i;
(e) transforming for each of the n discrete states, by the machine learning system, the current state x(t) and the action α(t) to a state change rate r_{θ}, where θ is a parametric weight value mapped by the machine learning platform to the current state x(t) and the action α(t) for each of the n discrete states;
(f) receiving, by a mechanistic platform, the state change rate r_{θ};
(g) calculating, by the mechanistic platform, a state differential rate dx/dt for each of the n discrete states, including a state differential rate dxᵢ/dt for the target cell type i, based on the state change rate r_{θ};
(h) integrating, by the mechanistic platform, the state differential rate dx/dt for each of the n discrete states from an initial time t₀ to the time τ to calculate the yield of the target cell type i at time τ; and
(i) outputting the calculated yield of the target cell type i for the time τ.

2. The computer-implemented method of claim 1, the method further comprising:
displaying the calculated yield on a display device.

3. The computer-implemented method of claim 1, the method further comprising:
optimizing the calculated yield under alternative actions α_{ALT}(t); and
outputting an optimized yield based on the alternative actions α_{ALT}(t).

4. The computer-implemented method of claim 3, wherein the optimizing comprises:
receiving, by the processor, one or more alternative actions α_{ALT}(t); and
repeating steps (d) to (i).

5. The computer-implemented method of claim 3, wherein the one or more alternative actions α_{ALT}(t) are received from a human interface device.

6. The computer-implemented method of claim 1, wherein the one or more measurements comprise:
(a) at least one of an inline process parameter or an online process parameter; and/or
(b) an online phenotypic measurement or an atline phenotypic measurement.

7. The computer-implemented method of claim 6, wherein the inline process parameter or the online process parameter comprises at least one of a pH measurement value, a temperature measurement value, a glucose measurement value, a lactate measurement value, a dissolved oxygen measurement value, a spectroscopy measurement value, a conductivity measurement value, an optical density measurement value, a capacitance measurement value, a medium viscosity measurement value, a redox potential measurement value, a mass spectrometry measurement value, and/or an ultrasound-based measurement value of fluid density.

8. (canceled)

9. The computer-implemented method of claim 6 [8], wherein the online phenotypic measurement or the atline phenotypic measurement comprises one or more images or flow cytometry.

10. The computer-implemented method of claim 1, wherein the stem cell culture:
(a) is selected from an embryonic stem cell culture, an adult stem cell culture, an induced pluripotent stem cell culture, or a trophoblast stem cell culture;
(b) is a mesenchymal stem cell culture, a hematopoietic stem cell culture, a neural stem cell culture, an epithelial stem cell culture, or a cord blood stem cell culture; and/or
(c) comprises progenitor cells.

11-12. (canceled)

13. The computer-implemented method of claim 10, wherein the progenitor cells are selected from the group consisting of mesodermal progenitor cells, endodermal progenitor cells, ectodermal progenitor cells, neural progenitor cells, cardiac progenitor cells, hematopoietic progenitor cells, mesenchymal stem cells, pancreatic progenitor cells, and a combination thereof.

14. The computer-implemented method of claim 1, wherein the stem cell culture undergoing differentiation results in the stem cell culture differentiating into a mesoderm, endoderm, and/or ectoderm.

15. The computer-implemented method of claim 14, wherein;
(a) the mesoderm comprises a skeletal muscle cell, a cardiac muscle cell, a kidney cell, a red blood cell, or a smooth muscle cell;
(b) the endoderm comprises a lung cell, a thyroid cell, or a pancreatic cell; and/or
(c) the ectoderm comprises a skin cell, a neuron cell, or a pigment cell.

16-17. (canceled)

18. The computer-implemented method of claim 1, wherein the yield of a target cell type comprises an amount of the target cell type, a level of growth of the target cell type, and/or a specific composition of the subpopulations of the target cell type.

19. The computer-implemented method of claim 1, wherein the discrete states of stem cells undergoing differentiation are selected from a specific cell type or subtype or a specific fate of the cell.

20. The computer-implemented method of claim 1, wherein the action is selected from the group consisting of maintaining the cell culture state, modulating the cell culture state, ending the cell culture, requesting further measurements, or requesting complementary measurements.

21. The computer-implemented method of claim 1, wherein the machine learning platform comprises a neural network.

22. The computer-implemented method of claim 1, wherein the mechanistic platform comprises:
(a) an application specific integrated circuit (ASIC); and/or
(b) one or more programs that are executed by the processor.

23. (canceled)

24. A non-transitory computer readable storage medium storing one or more programs, the one or more programs comprising instructions, which, when executed by a processor, perform:
(a) receiving, by the processor, a request for a yield of a target cell type i at a specific time τ in the future;
(b) receiving, by the processor, one or more measurements of a cell culture containing n discrete states of cells undergoing cell differentiation at time t, where n is a positive integer;
(c) providing, by the processor, the one or more measurements of the cell culture to a machine learning platform;
(d) generating, by the machine learning platform, a mathematical representation for each of the n discrete states based on the one or more measurements, the mathematical representation including a current state x(t) and an action α(t) for each of the n discrete states at time t, including a current state xᵢ(t) and an action αᵢ(t) for the target cell type i;
(e) transforming for each of the n discrete states, by the machine learning system, the current state x(t) and the action α(t) to a state change rate r_{θ}, where θ is a parametric weight value mapped by the machine learning platform to the current state x(t) and the action α(t) for each of the n discrete states;
(f) receiving, by a mechanistic platform, the state change rate r_{θ};
(g) calculating, by the mechanistic platform, a state differential rate dx/dt for each of the n discrete states, including a state differential rate di/dt for the target cell type i, based on the state change rate r_{θ};
(h) integrating, by the mechanistic platform, the state differential rate dx/dt for each of the n discrete states from an initial time t₀ to the time τ to calculate the yield of the target cell type i at time τ; and
(i) outputting the calculated yield of the target cell type i for the time τ.

25. The non-transitory computer readable storage medium of claim 24, wherein the one or more programs comprise instructions, which, when executed by the processor, perform:
(a) displaying the calculated yield on a display device;
(b) optimizing the calculated yield under alternative actions α_{ALT}(t); and
outputting an optimized yield based on the alternative actions α_{ALT}(t); or
(c) receiving, by the processor, one or more alternative actions α_{ALT}(t); and
repeating steps (d) to (i).

26-27. (canceled)

28. The non-transitory computer readable storage medium of claim 24, wherein the one or more alternative actions α_{ALT}(t) are received from a human interface device.

29. The non-transitory computer readable storage medium of claim 24, wherein the one or more measurements comprise:
(a) at least one of an inline process parameter or an online process parameter; and/or
(b) an online phenotypic measurement or an atline phenotypic measurement.

30. The non-transitory computer readable storage medium of claim 29, wherein the inline process parameter or the online process parameter comprises at least one of a pH measurement value, a temperature measurement value, a glucose measurement value, a lactate measurement value, a dissolved oxygen measurement value, a spectroscopy measurement value, a conductivity measurement value, an optical density measurement value, a capacitance measurement value, a medium viscosity measurement value, a redox potential measurement value, a mass spectrometry measurement value, and/or an ultrasound-based measurement value of fluid density.

31. (canceled)

32. The non-transitory computer readable storage medium of claim 29, wherein the online phenotypic measurement or the atline phenotypic measurement comprises one or more images or flow cytometry.

33. The non-transitory computer readable storage medium of claim 24, wherein the stem cell culture:
(a) is selected from an embryonic stem cell culture, an adult stem cell culture, an induced pluripotent stem cell culture, or a trophoblast stem cell culture;
(b) is a mesenchymal stem cell culture, a hematopoietic stem cell culture, a neural stem cell culture, an epithelial stem cell culture, or a cord blood stem cell culture; and/or
(c) comprises progenitor cells.

34-35. (canceled)

36. The non-transitory computer readable storage medium of claim 33, wherein the progenitor cells are selected from the group consisting of mesodermal progenitor cells, endodermal progenitor cells, ectodermal progenitor cells, neural progenitor cells, cardiac progenitor cells, hematopoietic progenitor cells, mesenchymal stem cells, pancreatic progenitor cells, and a combination thereof.

37. The non-transitory computer readable storage medium of claim 24, wherein the stem cell culture undergoing differentiation results in the stem cell culture differentiating into a mesoderm, endoderm, and/or ectoderm.

38. The non-transitory computer readable storage medium of claim 37, wherein:
(a) the mesoderm comprises a skeletal muscle cell, a cardiac muscle cell, a kidney cell, a red blood cell, or a smooth muscle cell;
(b) the endoderm comprises a lung cell, a thyroid cell, or a pancreatic cell; and/or
(c) the ectoderm comprises a skin cell, a neuron cell, or a pigment cell.

39-40. (canceled)

41. The non-transitory computer readable storage medium of claim 24, wherein the yield of a target cell type comprises an amount of the target cell type, a level of growth of the target cell type, and/or a specific composition of the subpopulations of the target cell type.

42. The non-transitory computer readable storage medium of claim 24, wherein the discrete states of stem cells undergoing differentiation are selected from a specific cell type or subtype or a specific fate of the cell.

43. The non-transitory computer readable storage medium of claim 24, wherein the action is selected from the group consisting of maintaining the cell culture state, modulating the cell culture state, ending the cell culture, requesting further measurements, or requesting complementary measurements.

44. The non-transitory computer readable storage medium of claim 24, wherein the machine learning platform comprises a neural network.

45. The non-transitory computer readable storage medium of claim 24, wherein the mechanistic platform comprises:
(a) an application specific integrated circuit (ASIC); and/or
(b) one or more programs that are executed by the processor.

46. (canceled)

47. An apparatus for optimizing a stem cell differentiation based on real-time monitoring of the stem cell culture undergoing differentiation using live cell measurements, the apparatus comprising:
(a) one or more input devices;
(b) one or more output devices including a human interface device;
(c) a machine learning platform;
(d) a mechanistic platform including an integrator;
(e) one or more processors; and
(f) a memory storing one or more programs to be executed by the one or more processors, the one or more programs comprising instructions for:
receiving a request for a yield of a target cell type i at a specific time τ in the future;
receiving one or more measurements of a cell culture containing n discrete states of cells undergoing cell differentiation at time t, where n is a positive integer;
providing the one or more measurements of the cell culture to the machine learning platform;
generating, by the machine learning platform, a mathematical representation for each of the n discrete states based on the one or more measurements, the mathematical representation including a current state x(t) and an action α(t) for each of the n discrete states at time t, including a current state x(t) and an action αᵢ(t) for the target cell type i;
transforming for each of the n discrete states, by the machine learning system, the current state x(t) and the action α(t) to a state change rate r_{θ}, where θ is a parametric weight value mapped by the machine learning platform to the current state x(t) and the action α(t) for each of the n discrete states;
receiving, by the mechanistic platform, the state change rate r_{θ};
calculating, by the mechanistic platform, a state differential rate dx/dt for each of the n discrete states, including a state differential rate de/dt for the target cell type i, based on the state change rate r_{θ};
integrating, by the integrator, the state differential rate dx/dt for each of the n discrete states from an initial time t₀ to the time τ to calculate the yield of the target cell type i at time τ; and
outputting the calculated yield of the target cell type i for the time τ.

48. The apparatus of claim 47, further comprising:
(g) a display device configured to display the calculated yield;
(h) a transmitter configured to send the calculated yield to a computing device; and/or
(i) a human interface device configured to receive one or more alternative actions α_{ALT}(t).

49. (canceled)

50. The apparatus of claim 47, wherein the one or more programs comprise instructions for:
(a) optimizing the calculated yield under alternative actions α_{ALT}(t); and
outputting an optimized yield based on the alternative actions α_{ALT}(t); and/or
(b) receiving the one or more alternative actions α_{ALT}(t) from a human interface device; and
repeating steps (d) to (i) to calculate the optimized yield based on the alternative actions α_{ALT}(t).

51-52. (canceled)

53. The apparatus of claim 47, wherein the one or more measurements comprise:
(a) at least one of an inline process parameter or an online process parameter; and/or
(b) an online phenotypic measurement or an atline phenotypic measurement.

54. The apparatus of claim 53, wherein the inline process parameter or the online process parameter comprises at least one of a pH measurement value, a temperature measurement value, a glucose measurement value, a lactate measurement value, a dissolved oxygen measurement value, a spectroscopy measurement value, a conductivity measurement value, an optical density measurement value, a capacitance measurement value, a medium viscosity measurement value, a redox potential measurement value, a mass spectrometry measurement value, and/or an ultrasound-based measurement value of fluid density.

55. (canceled)

56. The apparatus of claim 53 [55], wherein the online phenotypic measurement or the atline phenotypic measurement comprises one or more images or flow cytometry.

57. The apparatus of claim 47, wherein the stem cell culture:
(a) is selected from an embryonic stem cell culture, an adult stem cell culture, an induced pluripotent stem cell culture, or a trophoblast stem cell culture;
(b) is a mesenchymal stem cell culture, a hematopoietic stem cell culture, a neural stem cell culture, an epithelial stem cell culture, or a cord blood stem cell culture; and/or
(c) comprises progenitor cells.

58-59. (canceled)

60. The apparatus of claim 57, wherein the progenitor cells are selected from the group consisting of mesodermal progenitor cells, endodermal progenitor cells, ectodermal progenitor cells, neural progenitor cells, cardiac progenitor cells, hematopoietic progenitor cells, mesenchymal stem cells, pancreatic progenitor cells, and a combination thereof.

61. The apparatus of claim 47, wherein the stem cell culture undergoing differentiation results in the stem cell culture differentiating into a mesoderm, endoderm, and/or ectoderm.

62. The apparatus of claim 61, wherein:
(a) the mesoderm comprises a skeletal muscle cell, a kidney cell, a cardiac muscle cell, a red blood cell, or a smooth muscle cell;
(b) the endoderm comprises a lung cell, a thyroid cell, or a pancreatic cell; and/or
(c) the ectoderm comprises a skin cell, a neuron cell, or a pigment cell.

63-64. (canceled)

65. The apparatus of claim 47, wherein the yield of a target cell type comprises an amount of the target cell type, a level of growth of the target cell type, and/or a specific composition of the subpopulations of the target cell type.

66. The apparatus of claim 47, wherein the discrete states of stem cells undergoing differentiation are selected from a specific cell type or subtype or a specific fate of the cell.

67. The apparatus of claim 47, wherein the action is selected from the group consisting of maintaining the cell culture state, modulating the cell culture state, ending the cell culture, requesting further measurements, or requesting complementary measurements.

68. The apparatus of claim 47, wherein the machine learning platform comprises a neural network.

69. The apparatus of claim 47, wherein the mechanistic platform comprises:
(a) an application specific integrated circuit (ASIC); and/or
(b) one or more programs that are executed by the processor.

70. (canceled)
